(19) United States
(12) Patent Application Publication
(10) Pub. No.: US 2003/0170318 A1
(43) Pub. Date: Sep. 11, 2003

(54) Fluoride compositions and uses thereof

(76) Inventor: Gregory Gene Steiner, Honolulu, HI (US)

Correspondence Address: GODBEY GRIFFITHS REISS, 1001 BISHOP STREET, 2300 PAUAHI TOWER, HONOLULU, HI, 96813, US

(21) Appl. No.: 10/353,221

(22) Filed: Jan. 27, 2003

Related U.S. Application Data
(63) Continuation-in-part of application No. 09/952,466, filed on Sep. 14, 2001, now abandoned.
(60) Provisional application No. 60/236,951, filed on Sep. 29, 2000.

(51) Int. Cl. A61K 33/14

(52) U.S. Cl. 424/673; 424/675; 424/676

(57) ABSTRACT

The present invention relates to methods of treating and preventing cancer in a patient comprising administering a therapeutic amount of a composition comprising fluoride. Also, encompassed in the present invention are chemotherapeutic and chemopreventative compositions comprising fluoride or a pharmaceutically acceptable salt thereof.

### RELATED APPLICATION(S)

This application is a continuation-in-part of U.S. application Ser. No. 09/952,466, filed Sep. 14, 2001, which claims the benefit of U.S. Provisional Application No. 60/236,951, filed on Sep. 29, 2000. The entire teachings of the above applications are incorporated herein by reference.

### BACKGROUND OF THE INVENTION

Cancer chemotherapy is the treatment of cancerous lesions with the goal of slowing or stopping cancer cell division and destruction of cancer cells. Cancer chemotherapy commonly uses cytotoxic drugs that have adverse side effects. Therefore, there is a need for effective cancer chemotherapeutic compositions that are effective in treating cancerous lesions, while having minimal deleterious effects on patients.

### SUMMARY OF THE INVENTION

The present invention relates to cancer chemotherapeutic and chemopreventative methods and compositions comprising fluoride. The chemotherapeutic and chemopreventative methods and fluoride compositions described herein are advantageous for treating and preventing a diverse array of cancers including esophageal, small intestinal, colon, rectal, pancreatic, lung, melanoma, breast, uterine, ovarian, testicular, prostate, kidney, brain, non-Hodgkin's lymphoma, multiple myeloma and lymphoid leukemia. Significantly, it is believed that the compositions of the present invention will have minimal side effects in patients.

In one embodiment, the present invention is directed to a method of treating or preventing cancer in a patient, wherein the cancer is selected from the group consisting of esophageal, small intestinal, colon, rectal, pancreatic, lung, melanoma, breast, uterine, ovarian, testicular, prostate, kidney, brain, non-Hodgkin's lymphoma, multiple myeloma and lymphoid leukemia, comprising administering a therapeutic amount of a composition comprising fluoride or a pharmaceutically acceptable salt thereof. The present invention can also be utilized to treat or prevent all susceptible cancers. The present invention encompasses compositions comprising fluoride or a pharmaceutically acceptable salts of fluoride such as sodium fluoride, calcium fluoride, magnesium fluoride, potassium fluoride, aluminum fluoride, strontium fluoride and sodium monofluorophosphate. The composition can be a liquid, a solid, a gas or an inhalant.

Methods of administering the compositions of the present invention to a patient include oral, intravenous, intramuscular, intratumorally and topically. Alternately, the compositions of the present invention can be delivered via inhalation. The present invention encompasses administering to a patient an amount of a fluoride composition sufficient to: obtain a blood plasma fluoride level between 10 μM to about 12 μM; obtain a blood plasma fluoride level of at least 12 μM; induce apoptosis of a cancer cell; decrease cell proliferation of a cancer cell; increase the tumor suppressing activity of G protein complexes; or induce the tumor suppressing activity of GAP complexes. For example, the methods of the present invention disclosed herein can stimulate the tumor suppressing activity of GAP proteins. The present invention also encompasses administering a composition comprising between 1 mg to about 15 g of fluoride or a pharmaceutically acceptable salt thereof to the patient per day.

In one embodiment, the present invention is a method of treating or preventing cancer in a patient, wherein the cancer is selected from the group consisting of esophageal, small intestinal, colon, rectal, pancreatic, lung, melanoma, breast, uterine, ovarian, testicular, prostate, kidney, brain, non-Hodgkin's lymphoma, multiple myeloma and lymphoid leukemia, comprising administering a composition comprising about 75 mg of fluoride or a pharmaceutically acceptable salt thereof to the patient per day. In a particular embodiment of the present invention, the composition is administered intravenously. In another particular embodiment, the composition is administered to the patient orally. The present invention also encompasses administering to the patient the composition in three doses comprising 25 mg of fluoride or a pharmaceutically acceptable salt thereof.

The present invention also provides methods to maintain the blood plasma fluoride level of a patient at a target level. In one embodiment, the present invention is a method to maintain the blood plasma fluoride level between 10 μM and 12 μM in a patient comprising obtaining a blood sample from a patient and measuring the fluoride content of the blood sample. When the blood plasma fluoride level is below 10 μM, a therapeutic amount of a composition comprising fluoride or a pharmaceutically acceptable salt thereof is administered to the patient that is sufficient to raise the blood plasma fluoride level of the patient to between 10 μM and 12 μM. The present invention encompasses repeating one or more of the above steps to titrate the blood plasma fluoride levels of the patient to between 10 μM and 12 μM.

In another embodiment, the present invention is a method to maintain the blood plasma fluoride level of at least 12 μM in a patient comprising obtaining a blood sample from a patient and measuring the fluoride content of the blood sample. When the blood plasma fluoride level is below 12 μM, a therapeutic amount of a composition comprising fluoride or a pharmaceutically acceptable salt thereof is administered to the patient that is sufficient to raise the blood plasma fluoride level of the patient to at least 12 μM. The present invention encompasses repeating one or more of the above steps to titrate the blood plasma fluoride levels of the patient to at least 12 μM.

In another aspect, the present invention is directed toward methods of evaluating the effectiveness of fluoride compositions in treating cancer. In one embodiment, the present invention is a method to evaluate the effectiveness of fluoride compositions for treating cancer, comprising obtaining cancer cells from the patient and culturing the cancer cells under suitable conditions to maintain their viability. The cancer cells are contacted with a therapeutic amount of a composition comprising fluoride or a pharmaceutically acceptable salt thereof. The compositions are effective in treating cancer if the compositions induce apoptosis of cancer cells, decrease cancer cell proliferation or activate the tumor suppressing activity of the GAP complex.

A further aspect of the present invention relates to cancer chemotherapeutic and cancer chemopreventative compositions. The present invention encompasses cancer chemotherapeutic and cancer chemopreventative compositions comprising fluoride or a pharmaceutically acceptable salt thereof. In one embodiment, the present invention is a cancer therapeutic composition comprising an amount of fluoride or a pharmaceutically acceptable salt thereof sufficient to generate at least one of the following: obtain a blood plasma fluoride level between 10 μM and 12 μM, induce apoptosis of cancer cells, decrease cell proliferation of cancer cells, or activate G protein complex or GAP complex tumor suppressing activity.

The present invention also encompasses a method of treating cancer with compositions comprising fluoride or a pharmaceutically acceptable salt thereof and one or both of the following: calcium and vitamin D. In one embodiment, the present invention is a method of treating cancer, wherein the cancer is selected from the group consisting of esophageal, small intestinal, colon, rectal, pancreatic, lung, melanoma, breast, uterine, ovarian, testicular, prostate, kidney, brain, non-Hodgkin's lymphoma, multiple myeloma and lymphoid leukemia, comprising administering a composition comprising fluoride or a pharmaceutically acceptable salt thereof and calcium. In an alternate embodiment, the composition of the present invention includes vitamin D.

In an additional alternate embodiment the present invention is directed to a method of treating cancer with compositions comprising fluoride or a pharmaceutically acceptable salt thereof and one or both of the following: calcium and vitamin D. In one embodiment, the present invention is a method of treating cancer, wherein the cancer is selected from the group consisting of esophageal, small intestinal, colon, rectal, pancreatic, lung, melanoma, breast, uterine, ovarian, testicular, prostate, kidney, brain, non-Hodgkin's lymphoma, multiple myeloma and lymphoid leukemia, comprising administering a composition comprising fluoride, calcium or a pharmaceutically acceptable salt thereof and vitamin D.

### DETAILED DESCRIPTION OF THE INVENTION

The present invention relates, at least in part, to the Applicant's discovery of the inverse correlation between cancer incidence rates and fluoride concentration in the drinking water. The present invention provides methods and compositions comprising fluoride useful for chemotherapy and chemoprevention of susceptible cancers. As used herein, “chemotherapy” or “chemotherapeutic” method or composition refers to a method or a composition that is used to treat cancer or any other disease. Further, “chemoprevention” or “chemopreventative” refers to a method or composition that is useful in preventing cancer or any other disease. It is believed that the methods and compositions of the present invention will have minimal side effects. Therefore, the methods and compositions of the present invention are valuable for therapeutic regiments.

In one embodiment, the present invention is a method for treating cancer in a patient comprising administering a therapeutic amount of a composition comprising fluoride or a pharmaceutically acceptable salt thereof. The present method is useful for treating all susceptible cancers. As used herein, “susceptible” means that a patient's cancer will be arrested, ablated, inhibited or diminished upon treatment with the methods and compositions described herein. The compositions and methods of the present invention can also be used to treat or prevent cancer in non-human animals.

Methods of determining the efficacy of the methods described herein in the treatment of a patient's cancer are well know to the average oncologist. For example, a decrease in the number of leukemic cells in a patient's blood sample after treatment with the present invention would demonstrate the efficacy of disclosed methods and compositions.

In a preferred embodiment, the present invention is a method for treating or preventing cancer in a patient, wherein the cancer is selected from the group consisting of esophageal, small intestinal, colon, rectal, pancreatic, lung, melanoma, breast, uterine, ovarian, testicular, prostate, kidney, brain, non-Hodgkin's lymphoma, multiple myeloma, and lymphoid leukemia comprising administering a therapeutic amount of a composition comprising fluoride or a pharmaceutically acceptable salt thereof. As used herein, a “therapeutic amount” is defined as an amount of a composition used in the methods of the present invention. “Therapeutic amount” is also defined herein as the amount of a composition of the present invention that is sufficient to treat or prevent cancer or have a desirable effect for the patient.

Pharmaceutically acceptable salts of fluoride compositions useful in the present invention include but are not limited to sodium fluoride, calcium fluoride, magnesium fluoride, potassium fluoride, aluminum fluoride, strontium fluoride and sodium monofluorophosphate. Additional “pharmaceutically acceptable salts” of fluoride that are appropriate for administering to a human are well known to one skilled in the art.

The compositions of the present invention can be a liquid, a solid, a gas or an inhalant. Preferably, the composition is either a solid (e.g. tablet) or a liquid (e.g. for use intravenously). The compositions can be administered to the patient orally, intravenously, intramuscularly, intratumorally or topically.

Preferably, the compositions of the present invention are administered orally or intravenously. Other methods of administering chemotherapeutic and chemopreventative compositions are known to medical clinicians.

In one embodiment, the present invention is a method of treating or preventing cancer in a patient comprising administering a composition comprising between 1 mg to about 15 g of fluoride to a patient per day. In a preferred embodiment, the present invention is a method of treating or preventing cancer in a patient comprising administering a composition comprising 75 mg of fluoride to a patient per day. In another preferred embodiment, the composition is administered in three doses comprising 25 mg of fluoride per day.

In one embodiment, the compositions of the present invention are administered to a patient less than once a day. In an alternate embodiment, the compositions of the present invention are administered to a patient at least once a day. In still another embodiment, the compositions of the present invention are administered to a patient continuously. The duration of the treatment will vary according to the health of the patient, the response of the patient to the methods of the present invention and other clinically relevant factors. It is well within the abilities of one skilled in the art to determine the appropriate duration of treatment.

It has been established that the fluoride ion replaces the hydroxyl ion in bone apatite during bone mineralization (Newman, W F and Newman, M W The Chemical Dynamic of Bone Mineralization. Chicago, Ill.: University of Chicago Press, Chicago, 1958). In general, the bones of a person who has had the proper nutritional level of fluoride throughout their lifetime are “packed” with fluoride. As used herein, “packed” refers to an amount of fluoride in the bone apatite adequate to provide a fluoride reservoir for the human body. It is believed that the bones serve as a fluoride reservoir and secrete fluoride into the blood to maintain health. When the proper blood plasma fluoride level is reached the excess fluoride is excreted in the urine. Conversely, when a person is deficient in fluoride (i.e. bones are not “packed” with fluoride) and consumes fluoride it is absorbed by the bones and the blood plasma fluoride level drops.

As disclosed herein, seventeen cancers are associated with fluoride deficiency (See Example 1). The present invention utilizes compositions comprising fluoride to treat or prevent these and other cancers. Dosage of the fluoride compositions of the present invention suitable for treatment of a patient depends on various factors such as a patient's age, state of health, cancer risk factors, type and severity of cancer, and genotype.

Chemotherapy, as routinely performed at present, consists of administering an amount of a composition to the patient that is estimated to produce the desired result. In regards to a dosage for preventing cancer, in general, an adult with low risk factors for cancer living in an area with minimal fluoride in the drinking water or food should consume an amount of fluoride per day to maintain a blood plasma fluoride level of about 5-10 μM. It is believed that a dose of 25 mg of fluoride per day may achieve such a blood plasma fluoride level but each patient should be titrated to ensure that the target blood plasma fluoride level is achieved. An adult living in a similar area with higher risk factors should consume an amount of fluoride sufficient to maintain a target blood plasma fluoride level of about 10-12 μM. It is believed that a patient that consumes about 75 mg of fluoride per day may achieve this target blood plasma fluoride level.

In general, the appropriate dosage of the compositions of the present invention for each patient should be titrated to ensure that the target blood plasma fluoride level is achieved and maintained for the duration of the therapy. As used herein, a “target” level refers to a preferred or therapeutic level that is useful for treating or preventing a cancer by the methods of the present invention. As used herein, “titration” refers to adjusting the dosage (amount) of the fluoride composition to achieve the target blood plasma fluoride level. Due to the risk of developmental side effects with consumption of fluoride during early life, the present invention is not recommended for patients under 18 years of age.

In one embodiment, the present invention is a method for preventing or treating cancer in a patient comprising administering an amount of a composition comprising fluoride sufficient to obtain a blood plasma fluoride level between about 10 μM and about 12 μM. In another embodiment, the present invention is a method for preventing or treating cancer in a patient comprising administering an amount of a composition comprising fluoride sufficient to maintain a blood plasma fluoride level between about 10 μM and about 12 μM.

In regards to treating a patient with a cancer associated with a deficiency of fluoride (See Example 1), administering fluoride intravenously may be preferred. In this embodiment, administering the compositions comprising fluoride replaces the fluoride that should be secreted by the bones. As a general rule, chemotherapeutic amounts of compositions comprising fluoride should be administered to a patient that are sufficient to maintain a blood plasma fluoride level of about 10 μM to about 12 μM. However, the amount of fluoride given to a patient can be modified to maintain a desired blood plasma fluoride range or target level. Since many factors affect the blood plasma level of fluoride, each patient must be titrated.

Current chemotherapy regimens are often in excess of known toxic levels for patients. It is known that the toxic blood plasma fluoride level is about 15 μM. Therefore, administrating to a patient an amount of a composition comprising fluoride that results in a blood plasma fluoride level above the toxic level is comprehended as a therapeutic amount. In one embodiment, the present invention is a method of treating cancer in a patient comprising administering a therapeutic amount of a composition comprising fluoride sufficient to maintain a blood plasma fluoride level of at least 12 μM. The above are guidelines and are not meant to be limiting in any way. It is well within the abilities of one skilled in the art to assess risk factors and titrate blood plasma fluoride to different target levels or ranges without departing from the scope of the invention encompassed by the appended claims.

Cancer results from the proliferation, migration and metatasis of cancer cells. One aspect of the present invention relates to methods that can inhibit (partially or completely) cancer cell proliferation, cell migration and metastasis. In a related aspect, the present invention is a method that causes the apoptosis of cancer cells.

Furthermore, it is well established that cancer can be caused by the loss of a tumor suppressor gene or the attenuation of the tumor suppressing activity of a gene product. Examples of tumor suppressor genes include p53, DCC, APC, NF1, NF2, RB, and WT1. The present invention encompasses methods to induce and/or maintain the expression of a tumor suppressor gene or induce, maintain or enhance the tumor suppressing activity of a gene product. Gene products are defined as DNA, RNA or protein encoded by a gene. It is also known that G protein complexes and GAP protein complexes have tumor suppressing activities. In another aspect, the present invention relates to methods to induce, maintain or enhance G protein complex and GAP complex tumor suppressing activity.

In one embodiment, the present invention is a method of treating or preventing cancer in a patient comprising administering a therapeutic amount of a composition comprising fluoride or a pharmaceutically acceptable salt thereof sufficient to cause at least one of the following: generate a blood plasma fluoride level between about 10 μM and about 12 μM; obtain a blood plasma fluoride level of at least 12 μM; induce apoptosis of a cancer cell; inhibit cancer cell proliferation; inhibit cancer cell migration; inhibit cancer cell metastasis; induce or maintain the expression of a tumor suppressor gene; induce, enhance or maintain the tumor suppressing activity of a gene product; induce, enhance or maintain the tumor suppressing activity of a G protein complex, and induce, enhance or maintain the tumor suppressing activity of a GAP complex. In a preferred embodiment, the present invention is a method that maintains the blood plasma fluoride target level throughout the course of treatment. “Maintain”, as used in reference to blood plasma fluoride level, means that the daily, weekly, monthly or yearly average blood plasma fluoride level of a patient is approximately the target level.

As used herein, “generate” and “obtain” can be used interchangeably in regards to blood plasma fluoride levels. As used herein, “inhibit” is defined as partial inhibition or complete inhibition. As used herein, “inhibit” can also refer to a decrease. In a particular embodiment, the present invention is a method of treating or preventing cancer in a patient comprising administering a therapeutic amount of a composition comprising fluoride or a pharmaceutically acceptable salt thereof sufficient to induce, enhance or maintain the tumor suppressing activity of Rho GAP complex or Ras GAP complex.

The present invention also encompasses cancer therapeutic compositions comprising fluoride. In one embodiment, the present invention is a cancer therapeutic composition comprising an amount of fluoride or a pharmaceutically acceptable salt thereof sufficient to: obtain a blood plasma fluoride level between about 10 μM and about 12 μM; obtain a blood plasma fluoride level of at least 12 μM; induce apoptosis of a cancer cell; inhibit cancer cell proliferation; inhibit cancer cell metastasis; inhibit cancer cell migration; induce or maintain expression of a tumor suppressor gene or gene product; induce, enhance or maintain the tumor suppressing activity of a gene product; induce, enhance or maintain the tumor suppressing activity of a G protein complex; induce, enhance or maintain the tumor suppressing activity of a GAP protein complex; or induce the differentiation of a cell that will aid in the treatment of the cancer and/or recovery of the patient.

Another aspect of the present invention relates to maintaining the blood plasma fluoride level to a therapeutic target level. In one embodiment, the present invention is a method of maintaining the blood plasma fluoride level between about 10 μM and about 12 μM comprising obtaining a blood sample from a patient and measuring the fluoride content in the blood sample. The next step is to administer a therapeutic amount of a composition comprising fluoride or a pharmaceutically acceptable salt thereof if the blood plasma fluoride level is below 10 μM that is sufficient to raise the blood plasma fluoride level to between about 10 μM and about 12 μM. The above steps can be repeated as a means to monitor and maintain the blood plasma fluoride level to the therapeutic target range.

In another embodiment, the present invention is a method of maintaining the blood plasma fluoride level of at least 12 μM comprising obtaining a blood sample from a patient and measuring the fluoride content in the blood sample. The next step is to administer a therapeutic amount of a composition comprising fluoride or a pharmaceutically acceptable salt thereof if the blood plasma fluoride level is below 12 μM that is sufficient to raise the blood plasma fluoride level to at least 12 μM. The above steps can be repeated as a means to monitor and maintain the blood plasma fluoride level to the therapeutic target range. Methods of measuring the blood plasma fluoride level are known to one skilled in the art. For example, the fluoride level in the blood can be measured with a fluoride electrode as described in Patel et al. (Bone (1996) 6(19):651-655) and references contained therein.

The present invention also relates to methods of evaluating the efficacy of the fluoride compositions described herein in treating or preventing cancer. In one embodiment, the present invention is a method of evaluating fluoride compositions for their effectiveness in treating or preventing cancer comprising obtaining cancer cells from a patient and culturing the cancer cells under conditions suitable to maintain their viability. The cultured cancer cells are then contacted with a therapeutic amount of a composition comprising fluoride or a pharmaceutically acceptable salt thereof. The fluoride composition is an effective chemotherapeutic agent if it has at least one of the following effects on the cultured cancer cells: induces apoptosis of a cancer cell; inhibits cancer cell proliferation; inhibits cancer cell migration; inhibits cancer cell metastasis; induces or maintains expression of a tumor suppressor gene or gene product; induces, enhances or maintains the tumor suppressing activity of a gene product; induces, enhances or maintains the tumor suppressing activity of a G protein complex; induces, enhances or maintains the tumor suppressing activity of a GAP protein complex; or induces the differentiation of a cell that will aid in the treatment of the cancer and/or recovery of the patient. The use of cell lines to evaluate the effectiveness of compositions disclosed herein is also comprehended.

Methods of measuring cell proliferation, cell migration, metastasis, and apoptosis are well known and routine in the art. Tumor suppressor genes are well known in the art. Methods for measuring their gene expression is routine (See Sambrook, J, et al. Molecular Cloning: A Laboratory Manual, Cold Spring Harbor Laboratory Press. 1982; Ausubel, F M., et al. Current Protocols in Molecular Biology, Greene Publishing Assoc. and Wiley-Interscience. 1987, & Supp. 49, 2000, the teaching of which are incorporated herein by reference).

It has been determined that fluoride can enhance G protein complex and GAP protein activity (See Example 1). Additionally, G complex and GAP complex proteins are well represented in the art. Measuring G and GAP complex tumor suppressing activities are known to one of skill in the art (See Manning, D R. G proteins: Techniques of Analysis. CRC Press. 1999).

The present invention is further described below through examples which are not intended to be limiting.

### EXAMPLE 1

Chemoprevention has been described as the intervention with specific agents to prevent, inhibit or reverse carcinogenesis before malignancy. Chemotherapy is the treatment of cancerous lesions with the goal of slowing or stopping cancer cell division and spreading. Chemotherapy for cancer traditionally used cytotoxic drugs with the intent of killing the cancer cells before the host succumbed to the cancer or the chemotherapy.

Fluoride is a common component of chemopreventive and chemotherapeutic agents. However, fluoride has not been recognized as having chemoprotective or chemotherapeutic properties. Fluoride has not been proposed as the active ingredient in chemopreventive or chemotherapeutic agents.

The correlation between latitude and cancer incidence rates is well established and has been recognized for many years. A number of explanations have been proposed for the effect of latitude on cancer incidence rates but no explanation has received widespread acceptance. Increased solar radiation in lower latitudes with the associated increased production of vitamin D has been correlated with a reduction in cancer incidence rates (Garland, C F, et al. Ann N Y Acad Sci (1999) 889:107-19). However, a recent publication found no correlation between cancer incidence rates and solar radiation (Kampman, E, et al. Cancer, Causes and Control (2000) 11(5):459-66).

In a study on the relationship of latitude and pancreatic cancer, Kato et al. reported a strong correlation between latitude and pancreatic cancer mortality and a strong inverse correlation with the average temperature (Kato, I, et al. Jpn J Clin Oncol (1985) 15(2):403-13). In Japan, they found that there was no difference between urban and rural pancreatic cancer mortality even though there was a much higher fat intake in large cities. He concluded that there remains the possibility that factors related to latitude or average temperature rather than diet may be involved in the occurrence of pancreatic cancer.

Two studies provide evidence that increased water consumption reduces bladder and colorectal cancer. Michaud et al. studied the incidence of bladder cancer in 47,909 individuals over a 10-year period (Michaud, D S, et al. N Engl J Med (1999) 340(18): 1390-7). They found that the daily fluid intake was inversely correlated with the risk of bladder cancer. In conclusion, they stated that a high fluid intake is associated with a decreased risk of bladder cancer in men. In a hospital based case-control study Tang et al. found a strong inverse correlation between water intake and rectal cancer among men (Tang, R, et al. Int J Cancer (1999) 82:484-489). The same trend was found for women but was not significant.

Many carcinogens have been identified that produce an increased incidence of cancer (Begg, C B, et al. Am J Public Health (2001) 91(3):355-9). The discovery that fluoride reduces the incidence of caries was made in the early 1900's (American Dental Association http://www.ada.org/public/topics/fluoride). By the 1950's municipal water departments were beginning to add fluoride to the water supply (American Dental Association http://www.ada.org/public/topics/fluoride). As water was being fluoridated concerns that fluoride is carcinogenic were being voiced. This concern lead to studies to determine if fluoride in the drinking water is related to an increase in cancer incidence rates (Hoover, R N, et al. J Natl Cancer (1976) 57(4):757-68).

In 1974, Nixon and Carpenter published a paper comparing the standardized mortality ratios in relation to the amount of fluoride in the drinking water (Nixon, J M, and Carpenter, R G, Lancet (1974) 2(7888):1068-71). They reported finding a statistically significant negative correlation between the standardized mortality rate and the fluoride content of the water. Hoover et al. studied site-specific cancer mortality in the counties of Texas classified them into four groups by fluoride concentration of the drinking water (Hoover, R N, et al. J Natl Cancer (1976) 57(4):757-68). Consistent trends were found for cancers of the buccal cavity, pharynx (males only), esophagus (males and females), and skin cancer (females). All trends indicated a reduction in cancer incidence with increased fluoride concentration. Multiple regression analysis revealed a statistically significant inverse correlation with the fluoride variable in four out of 64 tests of significance. Doll and Kinlen studied cancer incidence between 1950 and 1970 in cities with fluoridated water and in cities without fluoridation. When account was taken for age, sex, and ethnic group the ratio between observed cancer mortality and expected cancer mortality fell slightly in the cities with fluoridated water and did not change in the non-fluoridated cities (Doll, R and Kinlen, L. Lancet (1977) 1(8025):1300-2).

Mcguire et al. in a case-control study of osteosarcoma patients found an inverse correlation between the fluoride concentration and the incidence of osteosarcoma (McGuire, S M, et al. J Am Dental Assoc (1991) 122(4):39-45). In 1995, Gelberg et al. published a case-control study comparing fluoride exposure and childhood osteosarcoma (Gelberg, K H, et al. American Journal of Public Health (1995) 85(12):1678-83). They found a statistically significant correlation between increased fluoride intake and a decrease in the incidence of osteosarcoma in males. They proposed that fluoride might have a protective effect in males.

In the 1970's, a series of studies were carried out on normal cells treated with various agents known to initiate mutations by inducing chromosomal damage. Vogel reported a strong antimutagenic effect of fluoride on mutation induced by Trenimon and 1-phenyl-3,3-dimethyltriazene in Drosophila (Vogel, E. Mutation Research (1973) 20:339-352). In 1973, Obe and Slacik-Erben reported similar findings as Vogel and proposed that sodium fluoride exerts its antimutagenic action by suppressing events leading to chromosomal breakage (Obe, G and Slacik-Erben, R. Mutation Research (1973) 19:369-371). In 1976, Slacik-Erben et al. reported that chromosomal aberrations induced by Trenimon revealed that pre, simultaneous and post-treatments with sodium fluoride significantly enhanced the frequency of undamaged mitosis (Slacik-Erben, R and Obe, G. Mutation Research (1976) 37:253-266). They interpreted their findings as an indication that sodium fluoride had significant antimutagenic activity.

Hirano et al. studied the effects of fluoride on cultures of the osteosarcoma cell line UMR 106. The addition of 0.5 mM fluoride resulted in the induction of apoptosis and a decrease in cell proliferation (Hirano, S and Ando, M. Arch Toxicol (1997) 72(1):52-8). Anuradha et al. reported that fluoride causes cell death in human leukemia (HL-60) cells by the activation of caspase-3 which in turn cleaves poly(ADP-ribose) polymerase leading to apoptosis (Anuradha, C D, et al. Arch Toxicol (2000) 74(4-5):226-30).

Endemic fluorosis is a disease caused by high fluoride consumption resulting from high concentrations of fluoride in drinking water or food (Singh, A. Punjab Medicine (1961) 42:229-246). Endemic fluorosis is identified by mottling of the teeth. Skeletal fluorosis can develop in areas with very high fluoride concentrations in the drinking water. In fluorosis, the fluoride ion replaces hydroxyl ions in bone apatite (Newman, W F and Newman, M W. The Chemical Dynamics of Bone Mineralization University of Chicago Press, Chicago, 1958).

Endemic fluorosis and the associated elevated fluoride in drinking water forms a fluoride belt which stretches across the north and east of Africa, through the middle east, across Pakistan and India, into Southeast Asia and the south of China. In many areas on this belt, endemic skeletal fluorosis has become a major health issue requiring the defluoridation of the drinking water. When plotted on the world map there appears to be an association between endemic fluorosis and reduced cancer incidence rates. The presence of endemic fluorosis allows us to identify areas with very high levels of fluoride in the drinking water. Latitude, temperature, and the fluoride concentration in the drinking water supply for areas with very high cancer incidence rates and areas with very low cancer incidence rates were compared in an effort to determine if cancer incidence rates are correlated with latitude, temperature, and fluoride concentration.

Results

Volume VII of Cancer Incidence in Five Continents was used as a reference to form the comparison groups of very high cancer incidence rate and very low cancer incidence rate (Parkin, D M, Whelan, S L, Ferlay, J, Raymond, L, Young, J, eds. Cancer Incidence in Five Continents. Volume VII. IARC Sci Publ (1997) (143):I-xxxiv,1-1240). Age-standardized rates for all sites but site 173 were chosen because many cancer-reporting stations do not fully report squamous cell and basal cell carcinoma of the skin (Weinstock, Mass., et al. Am J Public Health (1992) 82(2):278-81).

The very high cancer incidence rate group includes those cancer-reporting stations with a male cancer incidence rate over 300 per 100,000 and a female cancer incidence rate over 250 per 100,000 (Table 1). The cancer incidence rate for males and females were added together to provide a total cancer incidence rate for each reporting station. The very low cancer incidence rate group includes cancer-reporting stations with male and female cancer incidence rates under 160 per 100,000 (Table 2). Again, the cancer incidence rate for males and females was added together to provided a total cancer incidence rate.

TABLE 1
VERY HIGH CANCER INCIDENCE RATE GROUP
Age-standardized cancer incidence rate per 100,000 population¹

| Location | Male | Fem. | Total |
|---|---|---|---|
| San Francisco, Black | 465 | 286 | 751 |
| Detroit, Black | 464 | 278 | 742 |
| San Francisco, White | 408 | 305 | 713 |
| Atlanta, Black | 450 | 253 | 703 |
| New Zealand, Maori | 360 | 340 | 700 |
| Connecticut, Black | 425 | 273 | 698 |
| Detroit, White | 401 | 294 | 695 |
| Hawaii, White | 398 | 295 | 693 |
| New Orleans, Black | 418 | 271 | 689 |
| Los Angeles, Black | 425 | 262 | 687 |
| Seattle | 395 | 290 | 685 |
| Los Angeles, White | 373 | 304 | 677 |
| Italy, Trieste | 414 | 256 | 670 |
| New Orleans, White | 389 | 273 | 662 |
| Atlanta, White | 384 | 273 | 657 |
| Uruguay, Montevideo | 317 | 255 | 626 |
| Central Calif., White | 351 | 271 | 622 |
| Canada, Yukon | 326 | 295 | 621 |
| Iowa | 348 | 271 | 619 |
| New Mexico, White | 354 | 261 | 615 |
| Canada, Nova Scotia | 338 | 268 | 606 |
| Canada, Ontario | 326 | 261 | 587 |
| Canada, Manatoba | 325 | 261 | 586 |
| Canada, Prince Edward Is | 322 | 260 | 582 |
| Australia, South | 324 | 251 | 575 |
| Scotland, West | 317 | 256 | 573 |
| Austria, Tyrol | 313 | 252 | 565 |
| Australia, West | 312 | 252 | 564 |
| Scotland | 306 | 257 | 563 |
| Canada, British Columbia | 307 | 254 | 561 |

¹Parkin, D M, Whelan, S L, Ferlay, J, Raymond, L, Young, J, eds. Cancer Incidence in Five Continents. Volume VII. IARC Sci Publ 1997 (143): I-xxxiv, 1-1240.

TABLE 2
VERY LOW CANCER INCIDENCE RATE GROUP
Age-standardized cancer incidence rate per 100,000 population¹

| Location | Male | Fem. | Total |
|---|---|---|---|
| The Gambia* | 56 | 39 | 95 |
| India, Barshi etc. | 50 | 54 | 104 |
| Senegal, Dakar** | 76 | 75 | 151 |
| Algeria, Setif | 107 | 67 | 174 |
| India, Karunagappally | 108 | 80 | 188 |
| India, Trivandrum | 108 | 86 | 194 |
| Mali, Bamako | 125 | 90 | 125 |
| India, Bangalore | 98 | 118 | 216 |
| Israel, Non-Jews | 128 | 94 | 222 |
| Singapore, Indian | 106 | 123 | 229 |
| Kuwait, Kuwaitis | 104 | 126 | 230 |
| Viet Nam, Hanoi | 143 | 89 | 232 |
| India, Madras | 117 | 130 | 247 |
| India, Bombay | 131 | 125 | 256 |
| Singapore, Malay | 145 | 133 | 278 |
| Lima, Peru | 124 | 151 | 275 |
| Thailand, Chiang Mai | 144 | 153 | 297 |
| New Mexico, Am Indian | 151 | 148 | 299 |
| Uganda, Kyadondo | 155 | 146 | 301 |

*Data from an earlier edition of Cancer Incidence in Five Continents Vol VI 1992
**Data from an earlier edition of Cancer Incidence in Five Continents Vol IV 1982
¹Parkin, D M, Whelan, S L, Ferlay, J, Raymond, L, Young, J, eds. Cancer Incidence in Five Continents. Volume VII. IARC Sci Publ 1997 (143): I-xxxiv, 1-1240.

While there is no organized database for the world's drinking water supply, most nations and many scientific investigations have published data on the world's drinking water. The fluoride concentration at the same site can vary considerably according to whether the water source is surface water, shallow bore wells or deep bore wells. The concentration of fluoride can also vary significantly from village to village. For this reason obtaining an exact figure for fluoride concentration in the drinking water for a cancer incidence-reporting station is difficult. Fluoride content in the water is related to geologic formations. Areas located in the same geologic formations will have similar fluoride levels in the ground water supply. For this reason, if a reporting station did not have a published fluoride level then the fluoride concentration from an adjoining area was used for comparison.

Cancer incidence, latitude, temperature, and fluoride concentration are listed for the very high and very low cancer incidence rate groups (Tables 3 and 4). For those cancer-reporting stations with fluoridation, the fluoride concentration after the addition of fluoride was used for calculating the correlation coefficient. The average maximum average temperature for the cancer incidence reporting stations was selected because the daytime temperature should have the most significant effect on human physiology assuming people are insulated from the effects of nighttime temperatures.

TABLE 3
VERY HIGH CANCER INCIDENCE RATE WITH LATITUDE,
TEMPERATURE, AND FLUORIDE IN THE DRINKING WATER

| Location | Total CI¹ | Lat.¹ | Temp² | Nat. Fl* | Fluorosis** | Fluoridation |
|---|---|---|---|---|---|---|
| San Francisco, Black | 751 | 38 | 63 | <.4 mg/l | − | +³ |
| Detroit, Black | 742 | 42 | 58 | <.4 mg/l | − | +³ |
| San Francisco, White | 713 | 38 | 63 | <.4 mg/l | − | +³ |
| Atlanta, Black | 703 | 34 | 72 | <.4 mg/l | − | +³ |
| Maori | 700 | 40 | 59 | <.4 mg/l | − | +³ |
| Connecticut, Black | 698 | 41 | 60 | <.4 mg/l | − | +³ |
| Detroit, White | 695 | 42 | 58 | <.4 mg/l | − | +³ |
| Hawaii, White | 693 | 22 | 84 | <.4 mg/l | − | −⁴ |
| New Orleans, Black | 689 | 30 | 78 | <.4 mg/l | − | +³ |
| Los Angeles, Black | 687 | 34 | 73 | <.4 mg/l | − | −⁵ |
| Seattle | 685 | 47 | 59 | <.4 mg/l | − | +³ |
| Los Angeles, White | 677 | 34 | 73 | <.4 mg/l | − | −⁵ |
| Italy, Trieste | 670 | 45 | 63 | <.4 mg/l | − | −⁶ |
| New Orleans, White | 662 | 30 | 78 | <.4 mg/l | − | +³ |
| Atlanta, White | 657 | 34 | 72 | <.4 mg/l | − | +³ |
| Uruguay, Montevideo | 626 | 35 | 70 | <.4 mg/l | − | +³ |
| Central Calif., White | 622 | 36 | 76 | <.4 mg/l | − | +³ |
| Canada, Yukon | 621 | 63 | 40 | <.4 mg/l | − | +³ |
| Iowa | 619 | 42 | 60 | <.4 mg/l | − | +³ |
| New Mexico, White | 615 | 35 | 70 | <.4 mg/l | − | +³ |
| Canada, Nova Scotia | 606 | 49 | 48 | <.4 mg/l | − | +³ |
| Canada, Ontario | 587 | 45 | 54 | <.4 mg/l | +⁷ | +³ |
| Canada, Manatoba | 586 | 49 | 47 | <.4 mg/l | − | +³ |
| Canada, Prince Edward Is | 582 | 47 | 49 | <.4 mg/l | − | +³ |
| Australia, South | 575 | 35 | 70 | <.4 mg/l | − | +³ |
| Scotland, West | 573 | 57 | 52 | <.4 mg/l | − | −⁸ |
| Austria, Tyrol | 565 | 46 | 57 | <.4 mg/l | − | −⁹ |
| Australia, West | 564 | 32 | 75 | <.4 mg/l | − | +³ |
| Scotland | 563 | 56 | 53 | <.4 mg/l | − | −⁸ |
| Canada, British Columbia | 561 | 49 | 56 | <.4 mg/l | − | +³ |

*Data from an earlier edition of Cancer Incidence in Five Continents Vol VI 1992
**Data from an earlier edition of Cancer Incidence in Five Continents Vol IV 1982
¹Parkin, D M, Whelan, S L, Ferlay, J, Raymond, L, Young, J, eds. Cancer Incidence in Five Continents. Volume VII. IARC Sci Publ 1997 (143): I-xxxiv, 1-1240.
²Weatherbase.com.
³Fluoridation, Calgary Regional Health Authority, www.crha.org.
⁴Honolulu Board of Water Supply. www.hbws.org.
⁵Los Angeles Department of Water and Power. ladwp.com.
⁶Funari, E, et al. Biomed Environ Sci (1991) 4(4): 339-47.
⁷Brothwell, D J and Limeback H. Community Dent oral Epidemiol (1999) 27(2): 130-6.
⁸Attwood, D and Blinkhorn, A S. Lancet (1988) 2(8605): 266-7.
⁹Nell, A and Sperr, W. Wien Klin Wochenschr (1994) 106(19): 608-14.

TABLE 4
VERY LOW CANCER INCIDENCE RATE WITH LATITUDE,
TEMPERATURE AND FLUORIDE CONCENTRATION IN THE DRINKING
WATER

| Location | Total CI¹ | Lat.¹ | Temp² | Nat. Fl* | Fluorosis** | Fluoridation |
|---|---|---|---|---|---|---|
| The Gambia | 98* | 13 | 80 | 10 mg/l⁴ | +⁴ | −³ |
| Barshi, Paranda, Bhum | 104 | 18 | 91 | 10 mg/l⁵ | +⁵ | −³ |
| Senegal, Dakar | 151** | 13 | 80 | 10 mg/l⁴ | +⁴ | −³ |
| Algeria, Setif | 174 | 35 | 72 | 4.0 mg/l⁶ | +⁶ | −³ |
| India, Karunagappally | 188 | 9 | 86 | 5.1 mg/l^{7,15} | +⁷ | −³ |
| India, Trivandrum | 194 | 8 | 87 | 5.1 mg/l^{7,15} | +⁷ | −³ |
| Mali, Bamako | 215 | 13 | 92 | 10 mg/l⁴ | +⁴ | −³ |
| India, Bangalore | 216 | 13 | 83 | 9.1 mg/l^{7,15} | +⁷ | −³ |
| Israel, Non-Jews | 222 | 32 | 69 | 5.0 mg/l⁸ | +⁸ | −³ |
| Singapore, Indian | 229 | 1 | 87 | 0.7 mg/l⁹ | +⁹ | +⁹ |
| Kuwait, Kuwaitis | 230 | 29 | 90 | 0.4 mg/l¹⁶ | +¹⁰ | +³ |
| Viet Nam, Hanoi | 232 | 21 | 80 | unknown | unknown | unknown |
| India, Madras | 247 | 13 | 90 | 3.3 mg/l¹¹ | +¹¹ | −³ |
| India, Bombay | 256 | 19 | 88 | .32 mg/l¹¹ | +¹¹ | −³ |
| Singapore, Malay | 278 | 1 | 87 | 0.7 mg/l⁹ | +⁹ | +⁹ |
| Lima, Peru | 275 | 12 | 73 | unknown | unknown | unknown |
| Thailand, Chiang Mai | 297 | 16 | 89 | 5.0 mg/l¹⁷ | +¹² | —³ |
| New Mexico, Am Indian | 299 | 35 | 70 | 4.1 mg/l¹³ | +¹³ | —³ |
| Uganda, Kyadondo | 301 | 0 | 79 | 1.5 mg/l¹⁴ | +¹⁴ | —³ |

*Data from an earlier edition of Cancer Incidence in Five Continents Vol VI 1992
**Data from an earlier edition of Cancer Incidence in Five Continents Vol IV 1982
¹Parkin, D M, Whelan, S L, Ferlay, J, Raymond, L, Young, J, eds. Cancer Incidence in Five Continents. Volume VII. IARC Sci Publ 1997 (143): I-xxxiv, 1-1240.
²Weatherbase.com.
³Fluoridation, Calgary Regional Health Authority, www.crha.org.
⁴Pontie, M, et al. Sante (1996) 6(1): 27-36.
⁵Somvanshi, P R, et al. J Assoc Physicians India (1990) 38(3): 217-9.
⁶Poey, J, et al. Eur J Toxicol Envirn Hyg (1976) 9(3): 179-86.
⁷Gopalakrishnan, P, et al. Natl Med J India (1999) 12(3): 99-103.
⁸Mann, J, et al. Community Dent Oral Epidemiol (1990) 18(2): 77-9.
⁹Lo, G and Bagramain, R A. Community Dent Oral Epidemiol (1996) 24: 25-7.
¹⁰Vigild, M, et al. Community Dent Health (1996) 13(1): 47.
¹¹Banerjee, S, ed. Fluorosis In India. Institute of Social Sciences (New Delhi, India), Manuscript report 7 1992.
¹²Phantumvanit, P, Songpaisan Y, Moller I J. A Defluoridator for Individual Households. World Health Forum Vol. 9 1988.
¹³Shannon, I L. N M Dent J (1970) 21(2): 10-1.
¹⁴Rwenyonyi, C, et al. Caries Res (1999) 33(4): 267-74.
¹⁵Susheela, A K. Epidemiology and control of fluorosis in India. www.NutritionFoundation.org/archives/apr84c.htm
¹⁶al-Dashti, A A, et al. Community Dent Health (1995)12(1): 42-7.
¹⁷Phantumvanit, P. Osterreichische Zeitschrift fur Stomalogie (1982) 79: 303-4.

The average latitude for the very high cancer incidence rate group is 41 degrees. The average latitude for the very low cancer incidence rate group is 16 degrees. The correlation between the cancer incidence rate and latitude is r=0.71.

The average maximum average temperature of the very high cancer incidence rate group is 63 degrees F. The average maximum average temperature of the very low cancer incidence rate group is 83 degrees F. The correlation between the cancer incidence rate and temperature is r=−0.87. The average fluoride concentration of the very high cancer incidence rate group is 0.71 mg/l. The average fluoride concentration of the very low cancer incidence rate group is 5.0 mg/l. The correlation between the cancer incidence rate and the fluoride concentration in the drinking water is r=−0.75.

The very high cancer incidence rate group seldom reported endemic fluorosis. Endemic fluorosis was reported in 17 of 19 very low cancer incidence reporting areas. Information could not be found on the fluoride concentration or the incidence of fluorosis in Hanoi, Viet Nam or Lima, Peru.

Upon review of the available data, fluoride was found in high concentrations in virtually all very low cancer incidence rate areas and was found in low concentrations in all very high cancer incidence rate areas (Tables 3 and 4).

TABLE 5
AVERAGE CANCER INCIDENCE RATE FOR DIFFERENT
CANCERS OF THE VERY LOW CANCER INCIDENCE GROUP
AND THE VERY HIGH CANCER INCIDENCE GROUP¹

| | Cancer incidence | Cancer incidence rate |
|---|---|---|
| | rate Very Low | Very High |
| Cancer | incidence group | incidence group |
| Lip | .4 | 1.5 |
| Tongue | 1.6 | 1.7 |
| Salivary Gland | .4 | .7 |
| Mouth | 3.0 | 2.6 |
| Oropharynx | .9 | 1.3 |
| Nasopharynx | 1.6 | .5 |
| Hypopharynx | 1.6 | .9 |
| Pharynx | .3 | .4 |
| Esophagus | 2.7 | 4.5 |
| Stomach | 8.0 | 8.6 |
| Small Intestine | .2 | 1.0 |
| Colon | 3.5 | 23.1 |
| Rectum | 3.1 | 11.7 |
| Liver | 6.7 | 5.2 |
| Gallbladder | 1.5 | 2.4 |
| Pancreas | 2.3 | 7.3 |
| Nose | .6 | .5 |
| Larynx | 2.5 | 4.2 |
| Lung | 11.7 | 50.3 |
| Bone | .8 | .8 |
| Melanoma | .5 | 8.5 |
| Breast | 21.31 | 78.0 |
| Uterus | 2.8 | 16.9 |
| Cervix | 19.5 | 10.8 |
| Ovary | 4.5 | 10.8 |
| Testis | .8 | 4.1 |
| Prostate | 7.6 | 77.3 |
| Bladder | 2.9 | 11.3 |
| Kidney | 1.3 | 8.0 |
| Brain | 1.9 | 5.2 |
| Thyroid | 2.1 | 3.3 |
| Hodgkin's | 1.7 | 2.4 |
| Non-Hodgkin's Lymphoma | 3.9 | 10.1 |
| Multiple Myeloma | .9 | 3.9 |
| Lymphoid leukemia | 1.1 | 4.0 |
| Myeloid Leukemia | 1.7 | 3.1 |
| Monocytie leukemia | .0 | .2 |

¹Parkin, D M, Whelan, S L, Ferlay, J, Raymond, L, Young, J, eds. Cancer Incidence in Five Continents. Volume VII. IARC Sci Publ 1997 (143): I-xxxiv, 1-1240.

The data found that there was a strong inverse correlation between fluoride in the drinking water and the cancer incidence rate (−0.75). The cancers with a marked reduction in the cancer incidence rate which correlated with an elevation in fluoride consumption were cancers of the esophagus, small intestine, colon, rectum, pancreas, lung, melanoma, breast, uterus, ovary, testis, prostate, kidney, brain, non-Hodgkin's lymphoma, multiple myeloma, and lymphoid leukemia (Table 5).

Research has established a correlation between cancer incidence rates and the environmental factors latitude, temperature, and water consumption. As a general rule, cancer incidence rates increase with increasing latitude (Parkin, D M, Whelan, S L, Ferlay, J, Raymond, L, Young, J, eds. Cancer incidence in five continents. Volume VII. IARC Sci Publ 1997;(143):I-xxxiv,1-1240). As disclosed herein, an even stronger correlation is found between ambient temperature and the rate of cancer incidence. With the exception of Hawaii (the majority of drinking water in Hawaii lacks any detectable fluoride) none of the areas with high cancer incidence are found in the tropics (Table 1). Almost all of the areas with low cancer incidence are found in the tropics (Table 2). Humans are warm blooded and live with a very closely regulated internal temperature. A person in Senegal is the same temperature as a person in the Yukon. With this understanding, latitude and temperature may only be indirectly correlated with the cancer incidence rate.

The laborer in Senegal must perspire much more than a laborer in the Yukon to maintain the same body temperature. This fact suggests water consumption may be the reason latitude and temperature are related to cancer incidence rates. Because of a higher ambient temperature the person in lower latitudes will consume more water to maintain a stable body temperature. If a lower cancer incidence rate is correlated with increased water consumption, it is probable that the lower cancer incidence rate is more likely the result of something in the water.

As demonstrated herein, fluoride concentration in the drinking water is inversely correlated with the cancer incidence rate. Thus, fluoride as chemoprotective agent explains why the cancer incidence rate is correlated with latitude, temperature, and water consumption. It is believed that the warmer the climate, the more water is necessary to properly maintain body temperature through sweating. People living in lower latitudes would consume more water and therefore more fluoride. The finding that higher concentrations of fluoride are commonly found in the water in lower latitudes with warmer climates would accentuate the amount of fluoride consumed in lower latitudes.

New Mexico poses a unique comparison of drinking water fluoride concentration and the cancer incidence rate. The State of New Mexico has the distinction of being listed in both the very high cancer incidence rate group for non-Hispanic whites and also in the very low cancer incidence rate group for American Indians. With the exception of the major population centers of Albuquerque and Santa Fe, many areas in the state report the fluoride concentration in the drinking water to be over 1.5 mg/l (mg/l=ppm) (US Geological Survey, Washington D.C., 1967 Fluoride concentration in the United States). Many water samples report fluoride concentrations above 5 mg/l and concentrations up to 20 mg/l have been documented (NMED Drinking Water Bureau, New Mexico Department of Health, www.nmenv.state.nm.us/gwb/haflor.htm). The major population centers are located in areas of low fluoride with Albuquerque being fluoridated since 1974 (NMED Drinking Water Bureau, New Mexico Department of Health, www.nmenv.state.nm.us/gwb/haflor.htm). The population demographics historically were made up of the white population concentrated in the cities with low fluoride in the drinking water and the American Indian population located in the rural areas with high levels of fluoride in the drinking water. The non-Hispanic white population of New Mexico has a combined male and female cancer incidence rate of 615 per 100,000 people. The American Indian population of New Mexico has a combined male and female cancer incidence rate of 299 per 100,000 individuals.

Children retain twenty percent of all fluoride consumed (Zohouri, F V and Rugg-Gunn, A J. Br. J Nutr (2000) 83(1):15-25). Because the body reabsorbs much of the fluoride released during bone remodeling the half-life of fluoride in the body has been estimated to be 20 years. The long half-life of fluoride in the body could help explain why some cultures retain their low cancer incidence rates when they move to areas with high cancer incidence rates. A person living the first 10 years of his life in a high fluoride area such as India or Southern China will feasibly maintain an elevated plasma fluoride level for the remainder of his life.

A factor affecting the fluoride intake of Asian populations is the fluoride found in tea (Lakdawala, D R and Puenkar, B D. Dent. Dialogue (1974) 1(3):16-22). Even in soils with low fluoride content the tea plant has the unique ability to concentrate fluoride in its leaves. Lakdawala et al. analyzed the fluoride concentration of different foods and found tea to contain higher fluoride concentrations than any other food (Lakdawala, D R and Puenkar, B D. Dent. Dialogue (1974) 1(3):16-22). Tea leaves high in fluoride have been found to cause fluorosis when both the drinking water and the diet are low in fluoride (Cao, J, et al. Food Chem Toxicol (1997) 35(8):827-33).

Bombay is located in a State with endemic fluorosis. However Bombay has very low cancer incidence and very low fluoride in the drinking water. With low fluoride in the drinking water and a very low cancer incidence rate Bombay tends to dispel fluoride as a chemoprotective agent. However, Lakdawala et al. studied the fluoride content of the food and water consumed in Bombay and found high concentrations of fluoride in the food supply as a result of being grown in areas with high fluoride concentrations in the water (Lakdawala, D R and Puenkar, B D. Dent. Dialogue (1974) 1(3):16-22).

Many potential chemoprotective agents such a lutein, EGCG, kava, and vitamin D have been studied. (Vainio, H and Rautalahti, M. Cancer Epidemiol Biomarkers Prev (1998) 7(8):725-8; Fujiki, H, et al. Proc Soc Exp Biol Med (1999) 220(4):225-8; Steiner, G G. Hawaii Med J (2000) 59(11):420-2; Garland, C F, et al. Ann N Y Acad Sci (1999) 889:107-19). The present invention identifies fluoride as the agent responsible for reduced cancer incidence worldwide.

Mode of Action

Fluoride is known to significantly increase the bioactivity of molecules (Welch, J T and Eswarakrishman, S. Fluorine in bioorganic chemistry. John Wiley and Sons Inc. New York. 1991). Fluoride can activate enzymes by way of the guanine nucleotide-binding proteins (G proteins), as in the activation of adenylate cyclase and polyphosphoinositide phosphodiesterase (O'dell, B L and Sunde, R A, eds. Handbook of Nutritionally Essential Mineral Elements. Marcell Dekker Inc. New York. 1997). Fluoride is known to stimulate G proteins. The addition of 0.5 mM fluoride to cultures of the ostosarcoma cell line UMR 106 resulted in the induction of apoptosis and a decrease in cell proliferation (Hirano, S and Ando, M. Arch Toxicol (1997) 72(1):52-8). Fluoride causes cell death in human leukemia (HL-60) cells by the activation of caspase-3 which in turn cleaves poly(ADP-ribose) polymerase leading to apoptosis (Anuradha, C D, et al. Arch Toxicol (2000) 74(4-5):226-30). As we disclose herein, it is the increase or enhancement of the bioactivity of molecules, the stimulation of G proteins and possibly other as yet unknown mechanisms whereby fluoride acts as a chemopreventive and chemotherapeutic agent.

The factors that regulate the cell cycle and in particular the factors that regulate cell growth have only recently begun to be understood. Cells respond to the needs of the organism by reacting to the presence of molecules that come into contact with the cell's membrane. If the cell has a receptor to a molecule that contacts its membrane, then the cell membrane receptor will initiate a reaction that is transmitted into the cell directing it to perform a desired function. G proteins are a group of proteins that are responsible for signal transduction from the cell membrane to other parts of the cell and are involved in regulating cell growth.

G proteins (GTP-dependant signal transducers) are GTPases and cycle between their active GTP-bound form and their inactive GDP-bound form. Each G protein has intrinsic GTPase activity that hydrolyses bound GTP to GDP. In other words, G proteins have the intrinsic ability to convert GTP to GDP thereby automatically converting itself from the active GTP form into inactive GDP. However, since GTP is much more abundant than GDP in the cytoplasm there is a predilection for the G protein to bind GTP and remain in its active state. When the G protein hydrolyses GTP to GDP, the G protein returns to the cell membrane to await activation.

If the external signal is a growth hormone that activates G proteins to signal for cell division, the intrinsic GTPase activity of the G proteins provides a degree of self regulation of the signal prompting the cell to divide. However, hydrolysis of GTP to GDP is very slow. If the intrinsic GTPase hydrolysis of the cell were the only mechanism for regulation, then the cell would be over stimulated resulting in the potential development of a tumor.

Several different proteins bind to either the GTP-bound form of G protein or the GDP-bound form of the G protein and stimulate or inhibit either the hydrolysis of GTP to GDP or the dissociation of GDP. The proteins which help regulate the activity of G proteins are identified by various names, but the group that has received attention in relation to cancer are called GTPase activating proteins or GAPs.

GAPs are produced by tumor suppresser genes which regulate G proteins by facilitating the hydrolysis of GTP to GDP. GAPs combine with the active G protein (GTP-bound) and speed up the hydrolysis of GTP to GDP. Therefore, the GAPs are powerful inhibitors of G protein signal transduction. If an external molecule stimulates cell division by converting GDP-bound G protein (inactive) to GTP-bound G protein (active), the signal to proceed with cell division would be limited in the presence of GAPs.

In a normal cell, genes produce proteins such as G proteins and their GAPs that closely regulate the cell cycle. However, if a mutation occurs, the function of the proteins produced by these genes can lose the ability to properly regulate the cell cycle.

Two groups of G proteins called Ras and Rho have been extensively studied by molecular oncologists because they are highly oncogenic. It is estimated that mutations in Ras are associated with 30% of all human cancers (Bos, J L. Cancer Res (1989) 49(17):4682-9). Ras mutations are found in 90% of pancreatic cancers and 50% of colon cancers (Bos, J L. Cancer Res (1989) 49(17):4682-9). The Ras-G proteins are responsible for signaling cell growth. When a mutation occurs at a critical place on a Ras gene the Ras protein produced by this gene is left in the switched “on” mode and continually stimulates the cell to divide.

It has been found that a mutation in one oncogene is insufficient to produce cancer. If a mutation occurs in a gene that stimulates cell division and this switch is left “on”, this will unlikely produce cancer. However, if a mutation also occurs in a gene that is involved in the regulation of the mutated gene, then uncontrolled cell growth and cancer can occur. For cancer to develop, it requires a mutation in the gene that stimulates cell growth and a mutation in the gene that limits the gene that stimulates cell growth. If a G protein Ras mutation leaves the G protein in the “on” mode and the GAP molecule is mutated and is unable to hydrolyze Ras GTP to Ras GDP, cancer can occur.

The Rho family of G proteins are downregulated by an intrinsic GTPase, which is further downregulated by GTPase-activating proteins (GAPs). RhoGAPs contain a arginine residue that is involved in the catalysis of GTP to GDP. When the RhoGAP is normal, this protein will produce hydrolysis of RhoGTP to RhoGDP (38,000 fold) (Graham, D L et al. Biochemistry (1999) 1938(3):985-91). When RhoGAP is mutated with a substitution of the arginine residue to an alanine residue, the RhoGAP will only hydrolyze RhoGTP to RhoGDP (160 fold) (Graham, D L et al. Biochemistry (1999) 1938(3):985-91). This mutation significantly reduces the ability of RhoGAP to convert the active RhoGTP G protein to the inactive RhoGDP form.

RhoGAPs and RasGAPs form aluminum fluoride complexes with the RhoGDP and RasGDP, which represent analogues of the transition state of Rho and Ras hydrolysis (Scheffzek, K, et al. Science (1997) 277(5324):333-338; Hoffman, G R, et al. J Biol Chem (1998) 273(8):4392-4399; Mittal, R, et al. Science (1996) 273(5271):115-117; Ahmadian, M R, et al. FEBS Lett (1997) 408(3):315-318). Mutant RhoGAP effectively forms the transition state complex RhoGDP-AIF-RhoGAP. The mutation of arginine to alanine is overcome in the presence of aluminum fluoride (AIF) and mutant RhoGAP is as effective as normal RhoGAP in the hydrolyzing RhoGTP to RhoGDP (Graham, D L et al. Biochemistry (1999) 1938(3):985-91). For cancer to develop, multiple mutations must occur in the regulator genes. A mutation in a signaling G protein genes and a mutation in its GAP would likely result in cancer. However, if aluminum fluoride is present, mutated RhoGAP is restored to proper function and cancer is prevented.

With the knowledge that an alteration in the regulation of G proteins is associated with an estimated 30% of all cancers, the relationship of Ras G proteins and it's regulators has been studied extensively. In the 1990's, the structure of the Ras G protein and it's GAP was elucidated.

When Ras and it's GAP were mixed there was no binding of Ras/RasGAP to hydrolyze Ras GTP to Ras GDP. However, when aluminum fluoride, magnesium fluoride, or beryllium fluoride was added, the Ras/RasGAP complex readily formed. It was also found that for this reaction to occur it was necessary that free fluoride be available in solution. Ras and it's GAP fit together structurally and aluminum fluoride is located between the Ras and it's GAP. Fluoride is located at a critical location and is needed to produce the RasGAP complex. The ability of the GAP molecule to react with Ras has been shown to be critical for the proper control of the cell cycle, and without the proper functioning of this complex it has been shown that cancer can occur.

It is believed that adequate concentrations of fluoride are needed in the cell in order for the proper functioning of the tumor suppresser activity of the Ras and Rho GAP complex.

Therapeutic Levels

The skeleton functions as a reservoir for storing fluoride. This system allows the body to maintain a large reservoir of fluoride in order to maintain a constant level of plasma fluoride. Due to the ability of the skeletal system to absorb large quantities of fluoride, laboratory analysis of the fluoride plasma levels are required in order to titrate the patients to the desired plasma fluoride level.

The plasma fluoride levels for the majority of the population living in the United States ranges between 1.1 μM and 1.8 μM. Analysis of epidemiological, laboratory, and clinical studies have resulted in the determination that plasma fluoride levels need to be maintained between about 10 μM and about 12 μM for the prevention and treatment of cancer. Because fluoride is absorbed by the skeleton, laboratory analysis is required to determine if adequate levels of plasma fluoride is being achieved with the dose administered. As therapy progresses and fluoride in the skeleton increases, less administered fluoride is required to maintain adequate plasma fluoride levels due to the restored ability of the skeleton to maintain a higher level of plasma fluoride from its reserves. For the treatment of cancer, intravenous administration of sodium fluoride is advised with regular monitoring of the plasma fluoride levels.

The initiation of therapy for the treatment of active cancer is advised to be 75 mg sodium fluoride per day via 24 hour continuous drip. The dose can then be adjusted to reach the therapeutic rage of about 10 μM to 12 μM plasma fluoride. As with most chemotherapeutic agents, the fluoride dose can be increased as needed until clinical improvement is noted or signs of toxicity develop.

For the prevention of cancer, the dose required to achieve the target range of plasma fluoride of sodium fluoride is 25 mg orally three times per day. Eventually this dose can be adjusted as the skeletal stores are increased in order to maintain the target fluoride plasma levels of 10 μM to 12 μM.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method for treating cancer in a patient comprising administering a therapeutic amount of a composition comprising fluoride or a pharmaceutically acceptable salt thereof.

2. The method of claim 1, wherein the cancer is selected from the group consisting of esophageal, small intestinal, colon, rectal, pancreatic, lung, melanoma, breast, uterine, ovarian, testicular, prostate, kidney, brain, non-Hodgkin's lymphoma, multiple myeloma, and lymphoid leukemia.

3. The method of claim 1, wherein the pharmaceutically acceptable salt is selected from the group consisting of sodium fluoride, calcium fluoride, magnesium fluoride, potassium fluoride, aluminum fluoride, strontium fluoride and sodium monofluorophosphate.

4. The method of claim 1, wherein the composition is selected from the group consisting of a liquid, a solid, a gas, and an inhalant.

5. The method of claim 1, wherein the composition is administered orally, intravenously, intramuscularly, intratumorally or topically.

6. The method of claim 2, wherein between 1 mg/day to about 15 g/day of the composition is administered to the patient.

7. The method of claim 2, wherein about 75 mg/day of the composition is administered to the patient.

8. The method of claim 2, wherein three 25 mg doses of the composition is administered to the patient per day.

9. The method of claim 2, wherein the amount of said composition is sufficient to obtain a blood plasma fluoride level between about 10 μM to about 12 μM.

10. The method of claim 2, wherein the amount of said composition is sufficient to obtain a blood plasma fluoride level of at least 12 μM.

11. The method of claim 2, wherein the amount of said composition is sufficient to induce apoptosis of a cancer cell.

12. The method of claim 2, wherein the amount of said composition is sufficient to inhibit cell proliferation of a cancer cell.

13. The method of claim 2, wherein the amount of said composition is sufficient to inhibit cell migration of a cancer cell.

14. The method of claim 2, wherein the amount of said composition is sufficient to inhibit metastasis of a cancer cell.

15. The method of claim 2, wherein the amount of said composition is sufficient to enhance tumor suppressing activity of a G protein complex.

16. The method of claim 2, wherein the amount of said composition is sufficient to enhance tumor suppressing activity of a GAP complex.

17. The method of claim 6, wherein the amount of said composition is sufficient to obtain a blood plasma fluoride level between about 10 μM to about 12 μM.

18. The method of claim 6, wherein the amount of said composition is sufficient to induce apoptosis of a cancer cell.

19. The method of claim 6, wherein the amount of said composition is sufficient to inhibit cell proliferation of a cancer cell.

20. The method of claim 6,-wherein the amount of said composition is sufficient to enhance the tumor suppressing activity of a G protein complex.

21. The method of claim 6, wherein the amount of said composition is sufficient to enhance tumor suppressing activity of a GAP complex.

22. The method of claim 7, wherein the composition is administered intravenously.

23. The method of claim 7, wherein the composition is administered orally.

24. A method of preventing cancer in a patient, wherein the cancer is selected from the group consisting of esophageal, small intestinal, colon, rectal, pancreatic, lung, melanoma, breast, uterine, ovarian, testicular, prostate, kidney, brain, non-Hodgkin's lymphoma, multiple myeloma, and lymphoid leukemia, comprising administering a therapeutic amount of a composition comprising fluoride or a pharmaceutically acceptable salt thereof.

25. The method of claim 24, wherein between 1 mg/day to about 15 g/day of the composition is administered to the patient.

26. The method of claim 24, wherein about 75 mg/day of the composition is administered to the patient.

27. The method of claim 24, wherein the pharmaceutically acceptable salt is selected from the group consisting of sodium fluoride, calcium fluoride, magnesium fluoride, potassium fluoride, aluminum fluoride, strontium fluoride and sodium monofluorophosphate.

28. The method of claim 24, wherein the composition is selected from the group consisting of a liquid, a solid, a gas, and an inhalant.

29. The method of claim 24, wherein the composition is administered orally, intravenously, intramuscularly, intratumorally or topically.

30. The method of claim 24, wherein the amount of said composition is sufficient to obtain a blood plasma level of fluoride between about 10 μM to about 12 μM.

31. The method of claim 24, wherein the amount of said composition is sufficient to enhance tumor suppressing activity of a G protein complex.

32. The method of claim 24, wherein the amount of said composition is sufficient to enhance tumor suppressing activity of a GAP complex.

33. A method of maintaining a blood plasma fluoride level between about 10 μM to about 12 μM in a patient comprising:
a) obtaining a blood sample from a patient;
b) measuring the fluoride content in the blood sample;
c) administering a therapeutic amount of a composition comprising fluoride or a pharmaceutically acceptable salt thereof when the blood plasma fluoride level is below 10 μM, whereby the amount of the said composition is sufficient to raise the blood plasma fluoride level to between about 10 μM to about 12 μM.
d) repeating steps a) through c) such that the blood plasma fluoride level is maintained between about 10 μM and about 12 μM.

34. A method of evaluating fluoride compositions for their effectiveness in treating cancer comprising:
a) obtaining cancer cells from a patient;
b) culturing said cancer cells under conditions suitable to maintain their viability;
c) contacting said cancer cells with a therapeutic amount of a composition comprising fluoride or a pharmaceutically acceptable salt thereof;
wherein the composition has at least one of the following characteristics: induces apoptosis of said cancer cells; inhibits cell proliferation of said cancer cells; inhibits cell migration of cancer cells; enhances G protein complex tumor suppressing activity and enhances GAP complex tumor suppressing activity, said composition is effective in treating cancer.

35. A method of evaluating fluoride compositions for their effectiveness in treating cancer, wherein the cancer is selected from the group consisting of esophageal, small intestinal, colon, rectal, pancreatic, lung, melanoma, breast, uterine, ovarian, testicular, prostate, kidney, brain, non-Hodgkin's lymphoma, multiple myeloma, and lymphoid leukemia, comprising:
a) obtaining cancer cells from a patient;
b) culturing said cancer cells under conditions suitable to maintain their viability;
c) contacting said cancer cells with a therapeutic amount of a composition comprising fluoride or a pharmaceutically acceptable salt thereof;
wherein the composition has at least one of the following characteristics: induces apoptosis of said cancer cells; inhibits cell proliferation of said cancer cells; inhibits cell migration of cancer cells; enhances G protein complex tumor suppressing activity and enhances GAP complex tumor suppressing activity, said composition is effective in treating cancer.

36. A cancer therapeutic composition comprising an amount of fluoride or a therapeutic salt thereof sufficient to:
a) obtain a blood plasma fluoride level between about 10 μM to about 12 μM;
b) induce apoptosis of cancer cells;
c) inhibit cell proliferation of cancer cells;
d) inhibit cell migration of cancer cells;
e) enhance G protein tumor suppressing activity; or
f) enhance GAP. complex tumor suppressing activity

37. A cancer therapeutic composition comprising an amount of fluoride or a therapeutic salt thereof sufficient to obtain a blood plasma fluoride level between 10 μM to about 12 μM and at least one of the following:
a) induce apoptosis of cancer cells;
b) inhibit cell proliferation of cancer cells;
c) inhibit cell migration of cancer cells;
d) enhance G protein tumor suppressing activity;
e) enhance GAP complex tumor suppressing activity.
